(19) United States
(12) Patent Application Publication
(10) Pub. No.: US 2008/0274949 A1
(43) Pub. Date: Nov. 6, 2008

(54) Formulation of Albumin-Free Erythropoietin

(75) Inventors: Cheong Weon Cho, Gyeonggi-do (KR); Soon Kwan Chung, Gyeonggi-do (KR); Jeong Ku, Gyeonggi-do (KR); Jun Hee Cheon, Gyeonggi-do (KR); Tae Kun An, Jeollabuk-do (KR); Eun Kyung Jeon, Gyeonggi-do (KR); Jae Kyoung Ko, Incheon (KR); Tae Hyoung Kim, Gyeongsangnam-do (KR); Yeong Ok Beak, Gyeonggi-do (KR); Hea Ran Suh, Seoul (KR)

Correspondence Address: Casimir Jones, S.C., 440 Science Drive, Suite 203, Madison, WI, 53711, US

(73) Assignee: CJ CHEILJEDANG CORPORATION

(21) Appl. No.: 10/567,475

(22) PCT Filed: Jul. 27, 2004
(86) PCT No.: PCT/KR2004/001891

§ 371 Date: May 20, 2008

(30) Foreign Application Priority Data
Aug. 6, 2003 (KR) 10-2003-0054260

(51) Int. Cl. A61K 38/22

(52) U.S. Cl. 514/8

(57) ABSTRACT

Disclosed is a stable pharmaceutical solution preparation of erythropoietin (EPO), which includes a stabilizing agent not containing a blood-derived protein, thereby maintaining EPO activity for a prolonged period of time without the risk of viral contamination. The stable solution preparation further includes a non-ionic surfactant and a tonicity agent, thereby preventing EPO loss during storage and facilitating administration to the body.

### TECHNICAL FIELD

The present invention relates, in general, to a stable erythropoietin (hereinafter, referred to simply as “EPO”) solution preparation that is free from blood-derived protein and thus has long-term storage stability without the risk of viral contamination and ensures biological activity by including a stabilizing agent capable of replacing a blood-derived component, albumin or purified gelatin.

### BACKGROUND ART

EPO is a glycoprotein hormone that belongs to a family of cytokines including colony stimulating factors (CSFs), and is produced mainly in the kidney and to some extent in the liver. EPO plays a central role in producing mature erythrocytes by promoting the differentiation and proliferation of erythroid progenitor cells. Due to its role, EPO has various applications, including treatments of anemia associated with kidney diseases, anemia requiring bone marrow transplantation, and anemia associated with rheumatoid arthritis, cancer- or antitumor agent-related anemia, AIDS-related anemia, and is applied for treating patients suffering with aplastic anemia and chronic renal failure.

With an aim to treat the aforementioned diseases, drug design for supplying stable preparations of proteins such as EPO in the market requires that chemical and physical changes, such as hydrolysis, disulfide exchange reaction, denaturation, agglutination and adsorption, which are observed during preparation of pharmaceutical formulations, are suppressed, and that biological activity of protein drugs is maintained.

Therefore, efforts have been made to suppress chemical or physical changes of EPO preparations and maintain biological activity of EPO during long-term storage. To maintain biological activity of the EPO protein drug during long-term storage, stabilizing agents may be used in pharmaceutical preparations of EPO.

For example, U.S. Pat. No. 4,879,272 discloses a method of preventing EPO in an aqueous solution from being denatured and being adsorbed onto the inner surface of the wall of a container by employing human serum albumin, bovine serum albumin, lecithin, dextrans, ethylene oxide-propylene oxide copolymers, hydroxypropyl cellulose, methylcellulose, polyoxyethylene hydrogenated castor oils, polyethylene glycols, and the like. Also, this patent describes the relationship between concentration of human serum albumin as an adsorption inhibitor and EPO loss due to adsorption.

As in the reference patent, human or bovine serum albumin, purified gelatin and the like are typically used as, stabilizing agents for improving protein stability in conventional formulations of protein drugs. However, since human serum albumin is a blood product relying on donated blood for its supply, it is difficult to avoid the risk of viral contamination completely.

To overcome this problem, efforts were made to develop d method of stabilizing EPO as a protein drug without employment of blood-derived components. For example, U.S. Pat. No. 4,992,419 discloses a biocompatible, storage-stable EPO preparation comprising EPO; a physiologically compatible phosphate buffer; 5 to 50 g/L of urea; 1 to 50 g/L of an amino acid, which is selected from the group consisting of L-glycine, L-alanine, L-arginine, L-leucine, L-phenylalanine, L-glutamic acid, L-threonine and mixtures thereof; and 0.05 to 5 g/L of a non-ionic surfactant, which is a polymacrogol type, such as polyethylene sorbitan laurate, sorbitan trioleate and oleic acid polyglycol ether. This patent suggests that the EPO protein can be formulated into a storage-stable form without albumin.

In addition, U.S. Pat. No. 6,120,761 suggests a technique for preparing an EPO preparation that is free from heterogeneous protein such as human serum albumin or purified gelatin and maintains EPO in a stable form by employing an amino acid, such as leucine, serine, glutamic acid, arginine, histidine, and the like, as a stabilizing agent.

Another stable EPO preparation is disclosed in International Patent Application WO 00/61169, which comprises a pH buffering agent, a sorbitan mono-9-octadenoate polyoxy-1,2-ethanediyl derivative and an amino acid. In this application, a preferred pharmaceutical formulation of EPO comprises a combination of polysorbate 80 and glycine as stabilizing agents in a phosphate buffer system.

Other efforts were made to stabilize protein drugs other than EPO. For example, International Patent Application WO 00/48635 (EP 1 154 796) discloses an albumin-free lyophilized Factor VIII composition for treating hemophilia A caused by Factor VIII deficiency, which comprises a coagulation factor, Factor VIII, that serves as an antihemophiliac factor. This application states that pharmaceutical preparations of blood components can be prepared without albumin by using a stabilizing agent and a bulking agent, such as amino acids and sugars, in detail, by adding to the preparations the following components in addition to Factor VIII; 4% to 10% of a bulking agent selected from the group consisting of mannitol, glycine and alanine, or 2% to 6% of hydroxyethyl starch (hereinafter, is referred to simply as “HES”) as a bulking agent; 1% to 4% of a stabilizing agent selected from the group consisting of sucrose, trehalose, raffinose and arginine; 1 mM to 5 mM calcium salt; 100 mM to 300 mM sodium chloride; and a buffering agent for maintaining a pH of approximately 6 to 8.

In addition, as described in U.S. Pat. No. 5,358,708, methionine, histidine or mixtures thereof can be used as a stabilizing agent for stabilization and extension of storage lifetimes of protein drugs such as interferons, granulocyte-macrophage colony-stimulating factors (GM-CSFs) or interleukins.

U.S. Pat. No. 4,457,916 discloses a method for stabilizing tumor necrosis factor (TNF), which is a protein produced by macrophages, in an aqueous solution or in a powder form, which is based on the use of a stabilizing agent selected from a non-ionic surfactant, at least one substance selected from the group consisting of D-galactose, D-xylose, D-glucuronic acid, trehalose, dextran and HES, and mixtures thereof. By employing such a stabilizing agent selected from a non-ionic surfactant, a specific sugar or sugar-related compound and mixtures thereof, TNF can be formulated into an aqueous solution or powder that can be stored for a prolonged period of time without losing its activity and is stable upon freezing, thawing, lyophilization and pretreatment by heating.

However, when EPO preparations are lyophilized for their stabilization, lyophilization increases the risk of the above-mentioned physical and chemical problems. Even in the case that these problems are solved, lyophilization of EPO preparations entails another drawback, that of high production cost.

Thus, the present inventors were intended to invent a stable EPO solution preparation in an injectable form, which is free from blood-derived protein and thus free of the risk of viral contamination, has long-term storage stability and ensures the biological activity, by employing an EPO stabilizing agent capable of replacing the conventionally used stabilizing agent for protein preparations, albumin.

### DISCLOSURE OF THE INVENTION

The present invention provides an injectable, stable erythropoietin (EPO) solution preparation which maintains its activity for a prolonged period of time without the risk of viral contamination by employing a stabilizing agent not containing a blood-derived protein, the preparation comprising EPO, an albumin-free stabilizing agent, a non-ionic surfactant and a tonicity agent.

### BRIEF DESCRIPTION OF THE DRAWING

FIG. 1 is a graph showing the relationship between the residual rate of EPO and the concentration of hydroxyethyl starch (HES) after one-week storage at 40° C.

### BEST MODE FOR CARRYING OUT THE INVENTION

It is apparent to those skilled in the art that different proteins can be, due to their chemical differences, gradually inactivated at different ratios during storage and under different conditions.

That is, the positive effect of a substance used for protein stabilization on prolonging a storage period of proteins is not equal between different proteins. For this reason, the amount and type of a stabilizing agent used to achieve storage stability varies according to the types of proteins of interest. Also, when an identical stabilizing agent is used for different proteins, it has different effects in protein stabilization since proteins are changed in nature and concentration during storage.

Therefore, the present inventors intended to find a non-protein stabilizing agent that can stabilize EPO during storage to provide a stable EPO solution preparation capable of maintaining the biological activity of EPO for a prolonged period of time without the risk of viral contamination.

Leading to the present invention, the intensive and thorough research, conducted by the present inventors with an aim to achieve the above purpose, resulted in the finding that the use of HES and/or a specific amino acid as a stabilizing agent leads to an injectable, stable EPO solution preparation which is free from heterogeneous protein such as human serum albumin or purified gelatin and thus free from the risk of viral contamination, and which maintains the biological activity of EPO for a prolonged period of time.

The term “stable” or “stabilizing (agent)”, as used herein, is intended to mean that the loss of an active component is generally lower than 10% under specific storage conditions for a predetermined period of time. Typically, it will be appreciated that an EPO preparation is stable when maintaining the residual rate of EPO at 90% or higher, preferably about 95% for two years at 10° C., for six months at 25° C. or for one or two weeks at 40° C.

Storage stability of EPO and other protein drugs is important for ensuring accurate dose, as well as for inhibiting potential production of antigenic substances of EPO. It is to be appreciated that a loss of about 10% of EPO during the preparation and/or storage is acceptable upon substantial administration as long as EPO is not converted to antigenic compounds in a form of aggregates or fragments in a composition.

Hereinafter, the present invention will be described in more detail.

In an aspect, the present invention provides a stable EPO solution preparation comprising a therapeutically effective amount of EPO, an albumin-free stabilizing agent, a non-ionic surfactant and a tonicity agent.

In a preferred aspect, the present invention provides a stable EPO solution preparation comprising hydroxyethyl starch (HES) or a mixture of HES and an amino acid as a stabilizing agent.

In the above aspect, hydroxyethyl starch (HES) used in the present invention is preferably used in a concentration of 0.1% to 10%, and more preferably 0.1% to 3%.

The stable EPO solution preparation of the present invention may further comprise another stabilizing agent, an amino acid selected from among glutamic acid, glutamine, glycine or salts thereof, and mixtures thereof.

In addition, the non-ionic surfactant used in the stable EPO solution preparation of the present invention may be selected from among polyoxyethylene alkyl ethers, polyoxyethylene fatty acid esters, polyoxyethylene alkyl phenol ethers, sorbitan fatty acid esters, polyoxyethylene sorbitan fatty acid esters, sucrose fatty acid esters and polyoxyethylene-polyoxypropylene copolymers.

More preferably, the non-ionic surfactant may be selected from among polysorbate 20 and 80 and mixtures thereof.

In addition, the tonicity agent used in the EPO solution preparation of the present invention may be selected from among sodium chloride, mannitol, sorbitol and mixtures thereof.

More preferably, the tonicity agent is sodium chloride.

In a more preferred aspect, the stable EPO solution preparation according to the present invention is a solution preparation dissolved in a physiologically acceptable buffer known in the art.

More preferably, the buffer is injectable water.

It will be apparent to those skilled in the art that the above and other objects, features and other advantages of the present invention are more clearly understood from the following detailed description, examples and accompanying claims.

EPO used in the present invention is prepared from a natural or recombinant origin or both by any method. Natural EPO may be extracted from blood or urine. Recombinant EPO may be produced in cultures of mammalian cells transformed by genetic recombination.

EPO is contained in the EPO solution preparation of the present invention in a therapeutically effective amount. Typically, the therapeutically effective amount of EPO is about 2,000 to 10,000 international units (IU) in a single-use vial.

EPO stabilizing agents commonly used in the art, which are pharmaceutically preferred compositions not containing a blood-derived component such as albumin and gelatin, are exemplified by sugars including monosaccharides and polysaccharides, sugar alcohols, cyclitols, amino acids, inorganic salts, organic salts, sulfur-containing reducing agents, surfactants and chelating agents. Other useful stabilizing agents may include basic compounds such as arginine, guanidine or imidazole, polymers such as polyvinyl-pyrrolidone and polyethylene glycol, and dipeptides such as glycylglycine and glycyl-L-glutamic acid.

As EPO stabilizing agents, the sugars may include monosaccharides, e.g., mannose, glucose, fructose and xylose, and polysaccharides, e.g., lactose, maltose, sucrose, raffinose and dextran, and the sugar alcohols may include mannitol, sorbitol and glycerol. The cyclitols may include inositol.

In addition, the amino acids as EPO stabilizing agent, include L and D isomers of glycine, alanine, lysine, leucine, glutamic acid, aspartic acid, histidine, proline and tryptophan, and salts thereof.

The EPO solution preparation may further comprise an inorganic salt, e.g., sodium chloride, potassium chloride, calcium chloride, sodium phosphate, potassium phosphate, and sodium hydrogen carbonate; an organic salt, e.g., sodium citrate, potassium citrate and sodium acetate; and a sulfur-containing reducing agent, e.g., glutathione, thioctic acid, sodium thioglycolate, thioglycerol, α-momothioglycerol and sodium thiosulfate.

Useful surfactants include non-ionic surfactants which include block polymers with hydrophilic or hydrophobic moieties, e.g., polyoxyethylene alkyl ethers, polyoxyethylene fatty acid esters, polyoxyethylene alkyl phenol ethers, sorbitan fatty acid esters, polyoxyethylene sorbitan fatty acid esters and sucrose fatty acid esters; and polyoxyethylene-polyoxypropylene copolymers, polymer activators synthesized by graft polymerization.

The polyoxyethylene sorbitan fatty acid esters include polysorbate marketed under the trade name of Tween. Also, commercially available examples of polyoxyethylene-polyoxypropylene copolymers include those marketed under the trade names of Poloxamer or Pluronic.

The injectable, stable solution preparation of EPO according to the present invention comprises a major stabilizing agent, HES; another stabilizing agent, an amino acid; a tonicity agent selected from among sodium chloride, mannitol, sorbitol and mixtures thereof; a non-ionic surfactant selected from polysorbate 20, polysorbate 80 and mixtures thereof; and injectable water.

In more detail, HES used as a major stabilizing agent in the present invention is a highly branched polymer of glucose units, which is synthesized by alkaline hydroxyethylation of amylopectin. Unlike albumin used as a plasma volume expander, which is a monodispersed colloid with a molecular weight of 69,000 g/mole, HES is a polydispersed colloid in which 80% of the polymer has molecular weights of 30 to 2,400,000 g/mole. HES useful in the present invention is a medium molecular weight HES with a mean molecular weight 200,000 g/mole.

HES contained as a major stabilizing agent in the stable EPO solution preparation of the present invention is preferably used in a concentration of 0.1% to 10%, and more preferably 0.1% to 3%.

Another stabilizing agent used in the stable EPO solution preparation of the present invention includes amino acids and their salts such as sodium salts, potassium salts and hydrochlorides.

The preferred amino acids include glutamine, glycine, arginine, proline, glutamic acid, histidine, and essential amino acids including isoleucine, leucine, lysine, phenylalanine, methionine, threonine, tryptophan and valine, and salts thereof. The above-mentioned amino acids or salts thereof may be added singly or in combinations of two or more.

Particularly preferred amino acids as another stabilizing agent according to the present invention are L-glutamic acid, L-glutamine, L-glycine and salts thereof. These amino acids and their salts may be added singly or in combinations of two or more.

The amount of the amino acid added to the stable EPO solution preparation of the present invention ranges from about 1 to 20 mg/ml, and preferably about 2 to 10 mg/ml.

Preferred non-ionic surfactants useful in the solution preparation of the present invention are polysorbate 20, polysorbate 80 and mixtures thereof.

The tonicity agent used in the stable EPO solution preparation of the present invention may be selected from among sodium chloride, mannitol, sorbitol and mixtures thereof.

The stable EPO solution preparation of the present invention is typically adjusted to a pH of about 5.0 to 8.0. The preferred pH range is between about 6.0 and 7.0. A suitable pH condition may be achieved by using an aqueous buffer solution of a tonicity agent selected from among sodium chloride, mannitol, sorbitol and other corresponding substances.

The stable EPO solution preparation of the present invention may be typically contained in a sealed, sterilized plastic or glass container. The solution preparation of the present invention may be supplied as a prescribed dose in an ampoule, vial or disposable syringe, or in a multiple dose form such as a bag or bottle for injection.

In the present invention, as described in detail in the following examples, EPO solution preparations were subjected to severe, accelerated stability tests at 40° C. and 25° C. for a predetermined period of time. Thereafter, the residual rate of EPO in each of the EPO solution preparations was measured and utilized for selection and determination of the stabilizing agents.

In more detail, EPO solution preparations containing HES as a major stabilizing agent were prepared, and stored at 40° C. and 25° C. for a predetermined period of time. The EPO residual rate in each of the preparations was measured by reverse phase high performance liquid chromatography (RP-HPLC). As a result, the residual rate of EPO was found to be higher in the solution preparations containing HES, L-glutamine, L-glutamic acid and L-glycine than other solution preparations, thereby ensuring long-term stability of EPO.

The stabilizing agent according to the present invention, HES, is capable of replacing albumin, has a relatively mild toxicity, can be easily obtained at low cost, and is free from the risk of transfusion-transmitted diseases, thus being convenient to use. In this regard, the present inventors found the fact that the employment of HES that is injectable and generally used as a plasma volume expender, a suspending agent and an anti-freezing agent in the art leads to preparation of an EPO solution preparation having long-term stability.

A better understanding of the present invention may be obtained through the following examples which are set forth to illustrate, but are not to be construed as the limits of the present invention.

### EXAMPLE 1

### Effect of HES with Various Concentrations on EPO Stability

To prepare EPO solutions containing 0-3% (0-30 g/L) of HES without addition of a specific amino acid, 0-3% of HES was dissolved in 0.9 L water for injection and stirred at 70±5° C. for over 20 min and cooled to 35° C. Sodium chloride and polysorbate 80 were added to each of the cooled solutions and dissolved therein. Additional water for injection was added to each of the solutions to achieve a final volume of 1 L. After being stirred, the solutions were individually adjusted to pH 6.9. Then, each solution was filtered through a 0.22-mm membrane and supplemented with a predetermined amount of EPO. A type-I glass vial was filled with the resulting solution, thus yielding a stable sample. EPO was used in an amount ranging from 2,000 to 10,000 IU.

The prepared stable samples were stored at 40° C. for one week, and the residual rate of EPO was evaluated by RP-HPLC.

HES was found to have an effect of stabilizing EPO at proper concentrations, thus ensuring the long-term storage stability of EPO. The results are given in Table 1, below.

TABLE 1
Results of stability tests for EPO solution preparations
containing different concentrations of HES

| | Test Examples | | | | | |
|---|---|---|---|---|---|---|
| Composition (mg/mL) | 1 | 2 | 3 | 4 | 5 | 6 |
| Sodium chloride | 9 | 9 | 9 | 9 | 9 | 9 |
| Polysorbate 80 | 0.5 | 0.5 | 0.5 | 0.5 | 0.5 | 0.5 |
| Hydroxyethyl starch (HES) | — | 2.5 | 5 | 10 | 20 | 30 |
| Residual rate (%) of EPO | 86 | 86 | 89 | 95 | 94 | 92 |
| after 1-week storage at 40° C. | | | | | | |

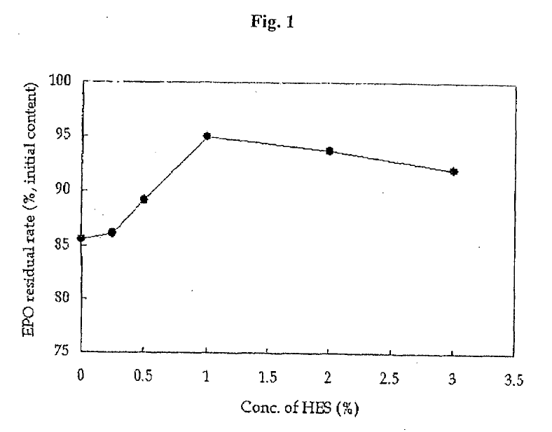

As shown in Table 1 and FIG. 1, higher residual rates of EPO were found in stable samples not containing a specific amino acid but HES in a concentration of 1% to 3%. The highest residual rate of EPO was observed in a 1% HES-containing sample.

### EXAMPLE 2

### Effect of Isotonicity of Solutions Containing HES and an Amino Acid on EPO Stability

Injectable EPO solution preparations were prepared according to the present invention using pure water. To render these solution preparations isotonic, 0.5 to 10 g/L of sodium chloride, mannitol, sorbitol or other corresponding substances were added to each of the solutions. As in Example 1, the solution preparations were adjusted to pH 6.9. To evaluate the effect of isotonicity of preparations on EPO stability, EPO solutions were prepared with a predetermined amount of HES and various amounts of sodium chloride, as shown in Table 2, below, according to the same method as in Example 1. Osmolarity was measured in each of the prepared samples using a freezing-point osmometer (Gonotec GmbH). The samples were stored at 40° C. for two weeks, and then evaluated for the residual rate of EPO by RP-HPLC. The results are given in Table 2, below.

TABLE 2
Results of stability tests for EPO solution preparations
containing different concentrations of NaCl

| | Test Examples | | | | |
|---|---|---|---|---|---|
| Composition (mg/mL) | 1 | 2 | 3 | 4 | 5 |
| Sodium chloride | 4.874 | 6.355 | 7.796 | 9 | 9.257 |
| Polysorbate 80 | 0.5 | 0.5 | 0.5 | 0.5 | 0.5 |
| Hydroxyethyl starch | 60 | 60 | 60 | 60 | 60 |
| (HES) | | | | | |
| Glycine | 5 | 5 | 5 | 5 | 5 |
| Osmolarity | 248 | 298 | 347 | 386 | 395 |
| Residual rate (%) of | 90 | 94 | 96 | 88 | 89 |
| EPO after 2-week | | | | | |
| storage at 40° C. | | | | | |

As shown in Table 2, when prepared with the NaCl concentrations of Test Examples 2 and 3, EPO solutions, which were measured to have osmolarities of 298 and 347 mOsm, were found to have relatively higher storage stabilities of 94% and 96%, respectively.

### EXAMPLE 3

### Evaluation of EPO Stability in a Solution Containing HES and Glutamine

To investigate EPO stability in a solution containing HES and glutamine, as stabilizing agents, an EPO solution was prepared with 1% HES and 8 mg/mL of glutamine according to the same method as in Example 1. The EPO solution was stored in an incubator at 40° C./RH75% for two weeks and in another incubator at 25° C./RH60% for six months. Then, the residual rates of EPO in the EPO solution were determined by the RP-HPLC method (Waters Company). The results are given in Table 3, below.

TABLE 3
Results of stability tests for the EPO solution
preparation containing HES and glutamine

| | | |
|---|---|---|
| Hydroxyethyl starch (HES) | 10 | mg/mL |
| Polysorbate 80 | 0.5 | mg/mL |
| Sodium chloride | 6.5 | mg/mL |
| Glutamine | 8 | mg/mL |

| | |
|---|---|
| Water for injection | Remainder |

Residual rate-of EPO after 2-week storage at 40° C. 91%
Residual rate of EPO after 6-month storage at 25° C. 95%

As a result, when the composition containing HES and glutamine was stored under the 40° C. and 25° C. severe conditions, it displayed 95% and 91% residual rates, respectively, compared to the initial content of EPO.

### EXAMPLE 4

### Evaluation of EPO Stability in a Solution Containing HES and Glutamic Acid

To investigate EPO stability in a solution containing HES and glutamic acid, as stabilizing agents, an EPO solution was prepared with 1% HES and 8 mg/mL of glutamic acid according to the same method as in Example 1. The EPO solution was stored in an incubator at 40° C./RH75% for two weeks and in another incubator at 25° C./RH60% for six months. Then, the residual rates of EPO in the EPO solution were determined by the RP-HPLC method (Waters Company). The results are given in Table 4, below.

TABLE 4
Results of stability tests for the EPO solution
preparation containing HES and glutamic acid

| | | |
|---|---|---|
| Hydroxyethyl starch (HES) | 10 | mg/mL |
| Polysorbate 80 | 0.5 | mg/mL |
| Sodium chloride | 6.5 | mg/mL |
| Glutamic acid | 8 | mg/mL |

| | |
|---|---|
| Water for injection | Remainder |

Residual rate of EPO after 2-week storage at 40° C. 94%
Residual rate Of EPO after 6-month storage at 25° C. 96%

As a result, when the composition containing HES and glutamic acid was stored under the 40° C. and 25° C. severe conditions, it displayed 94% and 96% residual rates, respectively, compared to the initial content of EPO.

### EXAMPLE 5

### Evaluation of EPO Stability in a Solution Containing HES, Glutamine and Glycine

To investigate EPO stability in a solution containing HES and two amino acids, glutamine and glycine, as stabilizing agents, an EPO solution was prepared with 1% HES, 8 mg/mL of glutamine and 2 mg/mL of glycine according to the same method as in Example 1. The EPO solution was stored in an incubator at 40° C./RH75% for two weeks and in another incubator at 25° C./RH60% for six months. Then, the residual rates of EPO in the EPO solution were determined by the RP-HPLC method (Waters Company). The results are given in Table 5, below.

TABLE 5
Results of stability tests for the EPO solution preparation
containing HES, glutamine and glycine

| | | |
|---|---|---|
| EPO | 2,000 | IU |
| Hydroxyethyl starch (HES) | 10 | mg/mL |
| Polysorbate 80 | 0.5 | mg/mL |
| Sodium chloride | 6.5 | mg/mL |
| Glutamine | 8 | mg/mL |
| Glycine | 2 | mg/mL |

| | |
|---|---|
| Water for injection | Remainder |

Residual rate of EPO after 2-week storage at 40° C. 96%
Residual rate of EPO after 6-month storage at 25° C. 95%

As a result, when the composition containing HES, glutamine and glycine was stored under the 40° C. and 25° C. severe conditions, it displayed 96% and 95% residual rates, respectively, compared to the initial content of EPO.

The foregoing examples are provided only to illustrate the present invention, and it should be understood that the present invention is not deemed to be limited thereto.

### INDUSTRIAL APPLICABILITY

As described hereinbefore, since the EPO solution preparation of the present invention is free from heterogeneous proteins such as human serum albumin or gelatin, it has excellent long-term storage stability without the risk of viral contamination.

1. An erythropoietin (EPO) solution preparation comprising:
a therapeutically effective amount of EPO; and
an albumin-free stabilizing agent which is selected from hydroxyethyl starch (HES) and a mixture of HES and an amino acid.

2. The EPO solution preparation according to claim 1, wherein the HES is contained in a concentration of 0.1% to 10%.

3. The EPO solution preparation according to claim 1, wherein the amino acid is selected from among glutamic acid, glutamine, glycine or salts thereof, and mixtures thereof.

4. The EPO solution preparation according to claim 1, further comprising a non-ionic surfactant and a tonicity agent.

5. The EPO solution preparation according to claim 4, wherein the non-ionic surfactant is selected from among polyoxyethylene alkyl ethers, polyoxyethylene fatty acid esters, polyoxyethylene alkyl phenol ethers, sorbitan fatty acid esters, polyoxyethylene sorbitan fatty acid esters, sucrose fatty acid esters and polyoxyethylene-polyoxypropylene copolymers.

6. The EPO solution preparation according to claim 4, wherein the tonicity agent is selected from among sodium chloride, mannitol, sorbitol and mixtures thereof.
